SEQUENCE LISTING (16 sequences)

SEQ ID NO: 1 - protein, 31 aa, synthetic construct
  REGION 1..31: note = Synthetic
SLNPEWNETKKGFYKKKQCRPSKGRKRGFCW

SEQ ID NO: 2 - protein, 30 aa, synthetic construct
  REGION 1..30: note = Synthetic
HDAPIGYDKKGFYKKKQCRPSKGRKRGFCW

SEQ ID NO: 3 - protein, 32 aa, synthetic construct
  REGION 1..32: note = Synthetic
AFNSYELGSDKKGFYKKKQCRPSKGRKRGFCW

SEQ ID NO: 4 - protein, 40 aa, synthetic construct
  REGION 1..40: note = Synthetic
RGVTEDYLRLETLVQKVVSKGFYKKKQCRPSKGRKRGFCW

SEQ ID NO: 5 - protein, 32 aa, synthetic construct
  REGION 1..32: note = Synthetic
ETFSDVWKLLKKGFYKKKQCRPSKGRKRGFCW

SEQ ID NO: 6 - protein, 25 aa, synthetic construct
  REGION 1..25: note = Synthetic
KGFYKKKQCRPSKGRKRGFCWSVDK

SEQ ID NO: 7 - protein, 25 aa, synthetic construct
  REGION 1..25: note = Synthetic
KGFYKKKQCRPSKGRKRGFCWAVDK

SEQ ID NO: 8 - protein, 21 aa, synthetic construct
  REGION 1..21: note = Synthetic
KGFYKKKQCRPSKGRKRGFCW

SEQ ID NO: 9 - protein, 21 aa, synthetic construct
  REGION 1..21: note = Synthetic
KGFYKRKQCKPSRGRKRGICW

SEQ ID NO: 10 - protein, 11 aa, synthetic construct
  REGION 1..11: note = Synthetic
GPTCAYGWCAK

SEQ ID NO: 11 - protein, 16 aa, synthetic construct
  REGION 1..16: note = Synthetic
CASSLNIASSLNIACK

SEQ ID NO: 12 - protein, 14 aa, synthetic construct
  REGION 1..14: note = Synthetic
CVALCREACGEGCK

SEQ ID NO: 13 - protein, 10 aa, synthetic construct
  REGION 1..10: note = Synthetic
CRPPRPARCK

SEQ ID NO: 14 - protein, 9 aa, synthetic construct
  REGION 1..9: note = Synthetic
CGFELETCK

SEQ ID NO: 15 - protein, 10 aa, synthetic construct
  REGION 1..10: note = Synthetic
CPGPEGAGCK

SEQ ID NO: 16 - protein, 10 aa, synthetic construct
  REGION 1..10: note = Synthetic
CGTEDAEDCK

(19) United States
(12) Patent Application Publication
(10) Pub. No.: US 2026/0265341 A1
(43) Pub. Date: Sep. 10, 2026

(54) EPISYNCHRONS FOR PRECISION MEDICINE

(71) Applicant: Desmond D Mascarenhas, Auburn, CA (US)

(72) Inventor: Desmond D Mascarenhas, Auburn, CA (US)

(21) Appl. No.: 18/718,024

(22) PCT Filed: Feb. 26, 2023
(86) PCT No.: PCT/US2023/013908

§ 371 Date: Jun. 8, 2024

(30) Foreign Application Priority Data
Mar. 2, 2022 (WO) PCT/US2022/018449

(51) Int. Cl. C07K 14/765; A61K 9/00; A61K 31/05; A61K 31/12; A61K 31/201; A61K 31/353; A61K 31/4741; A61K 38/00; A61K 47/30; C07K 1/107

(52) U.S. Cl. CPC C07K 14/765; A61K 9/0043; A61K 31/05; A61K 31/12; A61K 31/201; A61K 31/353; A61K 31/4741; A61K 38/00; A61K 47/30; C07K 1/1075; C07K 1/1077; C07K 2319/00

(57) ABSTRACT

This invention relates to the field of peptide diagnostics and therapeutics, and more particularly to novel structures and uses of a ligand-modified species of immodulin peptides herein termed “episynchrons”—end-modified immodulin peptides capable of driving the enhanced formation of RXR heterodimers in the nucleus of living mammalian cells—to modulate immunoregenerative processes, among other uses. The covalent addition of the exemplified nuclear receptor ligands to the carboxyterminus of a peptide prior to full chain elongation during peptide synthesis is novel and relevant to the cost of production of these agents at scale. Moreover, the facile and organelle-specific ‘on-demand’ reprogramming of transcriptional sets within target tissues is a technical breakthrough relevant to the future of precision medicine. Pre-designed tissue-specific epigenetic synchrony made possible by the unique structure of episynchrons in combination with the formulation of episynchrons in zip-coded microparticles or nanoparticles delivered in a location-specific manner, is a powerful and novel concept for twenty-first century medicine. The compositions and methods provided herein demonstrate new and surprising biological activities for episynchrons that are useful in treating a wide range of dysfunctions and perturbations in mammals in the context of organ- and stress-specific precision medicine.

### TECHNICAL FIELD

This invention relates to the field of peptide therapeutics, and more particularly to the novel structures and uses of a C-terminal ligand-modified class of immodulin peptides herein termed “episynchrons”. The compositions and methods provided demonstrate novel compositions and functions for these modified peptides. In light of their adjustable mechanistic specificity, episynchrons are powerful new tools in precision medicine, particularly when administered using routes and formulations optimized for targeted delivery. In combination with such routes and formulations, episynchrons may be used to treat dysfunctions in mammals with greater safety and specificity.

This invention claims priority benefit to PCT/US2022/018449 with an international filing date of 2 Mar. 2022, which claims benefit of international application number PCT/US2021/046814 with an international filing date of 20 Aug. 2021 and international application number PCT/US2021/021433 with an international filing date of 9 Mar. 2021. PCT/US2021/021433 claims priority to international application number PCT/US2020/024828 with an international filing date of 26 Mar. 2020.

Incorporation by Reference of Sequence Listing file: Sequence Listing is entered into the application as part of the disclosure.
Name of file: Mascarenhas_US23-13908-SEQLIST.xml
Date of Upload: Jun. 8, 2024
Size of TXT file in bytes: 20 KB

### SUMMARY OF THE INVENTION

Immodulin peptides are described in PCT/US2022/018449, PCT/US2021/046814, PCT/US2021/021433 and PCT/US2020/024828, as well as in scientific publications (see, for example, Mascarenhas, 2022 and references cited therein). The term ‘precision medicine’, as used herein, refers to highly specific and safe interventions.

This invention provides an industrially relevant method for covalent addition of nuclear receptor ligands to the C-terminal lysine residue of an immodulin peptide as a part of chain elongation during peptide synthesis, thereby generating a novel type of molecule herein termed an “episynchron”. Episynchrons comprising immodulin peptides have not been previously disclosed. The useful practical implications of episynchrons include the facilitation of novel strategies for on-demand RXR heterodimer formation in vivo, an aspect of significant relevance to twenty-first century precision medicine. Also provided herein are unexpected and useful biological activities of episynchrons.

PCT/US2020/024828 teaches methods and compositions based on N-terminal covalent addition of non-amino acid carboxylate small molecules to immodulin peptides, a conjugation step that occurs after assembly of the amino acid chain in synthesis. Covalent addition of small molecules to peptides and proteins is widely employed in laboratory research, but can be prohibitively expensive to scale in manufacture, as well as biologically problematic, for example, with respect to immunogenicity. Using amide bonds that resemble those naturally present in peptides and proteins for covalent addition of small molecules to peptides is therefore desirable. Sufficient molar ratios of a non-amino acid carboxylate must be used in N-terminal modification, in order to satisfy not just the desired reaction with the correctly assembled chain, but also the much more numerous failure sequences (such as deletions) accumulated during chain elongation during automated peptide synthesis. For modified immodulin peptides, which can often be over 40 amino acid residues in length, such failure sequences can significantly raise the cost of recovery of desired product from a reaction mixture. In practice, therefore, the use of the N-terminal addition method disclosed in PCT/US2020/024828 is largely limited by the cost of the non-amino acid carboxylate reagent employed.

The present invention, in contrast, teaches the covalent addition of non-amino acid carboxylates to a C-terminally located lysine in the immodulin peptide sequence. This reaction occurs at or near the start of amino acid chain elongation during synthesis (which, in automated peptide synthesis, is the C-terminus of the peptide), thereby providing homogeneous substrates during the covalent addition of a non-amino acid carboxylate to, say, the epsilon amine of a C-terminal lysine monomer. Regardless of the length of the final peptide chain, the option to couple a non-amino acid carboxylate to the epsilon amino group of a C-terminal lysine monomer via an covalent bond obviates many of the above-mentioned technical problems encountered, when using the method disclosed in PCT/US2020/024828. In practice, this vastly increases the options for adducts previously foreclosed due to cost i.e. C-terminal addition allows for the addition of more expensive compounds. The invention provides for the addition of non-amino acid carboxylates wherein said molecules can bind a nuclear receptor (NR) with sub-millimolar affinity in vitro, and many classes of NR-ligand adducts that would be cost-prohibitive for N-terminal addition become eligible for cost-efficient industrial use if C-terminal addition is used instead. On the other hand, there are other technical issues that can limit C-terminal in-synthesis conjugation of these non-amino acid carboxylates. For example, since they are added at the beginning of synthesis, the compounds must be stable enough to survive multiple cycles of synthesis under the harsh conditions typically employed for modern automated peptide synthesis. Moreover, for some compounds, if the conjugation is not sufficient efficient, the C-terminal monomer may need to be modified and pre-purified prior to use in chain elongation in order to avoid the very problems that C-terminal addition is designed to avoid. The general methods disclosed herein are applicable to a large number of compounds that are capable of binding a nuclear receptor with sub-millimolar affinity in vitro but are too expensive or otherwise impractical for use at scale by the method disclosed in PCT/US2020/024828. It is important to note, however, that it is not easy to predict in advance which ligands will work satisfactorily, and which will not. None of the nuclear-receptor-binding compounds successfully exemplified here have ever been previously coupled to the C-terminal lysine residue of an immodulin peptide (or, indeed, to the best of the inventor's knowledge, to any other peptide). This invention discloses, for the first time, small non-amino acid carboxylates—that can ligand NRs with sub-millimolar affinity in vitro—covalently and cost-effectively conjugated to the C-terminus of an immodulin peptide during synthesis. Such compositions are unprecedented.

A small number of off-the-shelf monomer reagents are available commercially which do pre-incorporate a small non-amino acid molecule such as biotin or FITC to an Fmoc lysine via a covalent bond formed with the epsilon amino group of the lysine. For example, Fmoc-Lys (biotin)-Wang monomer is widely available and can be purchased from multiple vendors. However, there are no commercially available reagents that pre-incorporate small lysine-bound non-amino acid molecules that bind a nuclear receptor with sub-millimolar affinity in vitro.

Unlike prior inventions, such as the methods disclosed in PCT/US2020/024828, the novel structures made possible by the method disclosed in this invention allows for the industrial use of relatively more expensive ligands of nuclear receptors, making a wider range of episynchrons amenable to the economics of synthesis at scale. In some exemplified cases, novel episynchrons enhance the formation of novel RXR heterodimers in vivo, as demonstrated by their biological activities. Some of the novel episynchrons disclosed herein exhibit surprising bioactivities in living mammalian cells. This invention provides examples of episynchrons that have novel utilities in medicine and cosmetics, among other domains.

This invention provides modified synthetic peptides. Peptide modifications include sequence changes and extensions to core sequences, substitution of amino acids with D-isomers, conjugation to bioactive small molecules, and complexation to metals, glycosaminoglycans, albumin, chitosan, transferrin, polymers or other helper molecules.

Immodulin peptides are believed to rapidly select and enter target cells and travel to specific cellular compartments (e.g. the nucleus), where they interact with cellular machinery in epigenetic ways. For example, they may bind transcriptional machinery and alter large transcriptional sets. A comprehensive description of immodulins can be found in PCT/US2020/024828, PCT/US2021/021433, PCT/US2021/046814, PCT/US2022/018449 and in a recent publication (Mascarenhas, 2022) as well as in references cited therein.

Retinoid X receptors (RXRs) are promiscuous partners in heterodimeric associations with other members of the Nuclear Receptor (NR) superfamily. RXR ligands (“rexinoids”) may transcriptionally activate heterodimers (such as RXRγ/Nur77, or RXRα/PPARα). RXRs are obligatory partners for a number of other NRs, giving RXRs a coordinating role at the crossroads of multiple signaling pathways. RXRs represent important targets for pharmacologic interventions and therapeutic applications. Receptor knockout studies demonstrate an important role for these receptors both during development and in adult differentiated tissues (cell proliferation, cell differentiation, cell death). These receptors also play an important regulatory role metabolic signaling pathways (glucose, fatty acid and cholesterol metabolism), including metabolic disorders such as type 2 diabetes, hyperlipidemia and atherosclerosis. RXRs function as master regulators producing diverse physiological effects through the activation of multiple nuclear receptor complexes. The retinoid X receptor (RXR) subgroup (NR2B) of NRs is composed of 3 members: RXRα (NR2B1), RXRβ (NR2B2), and RXRγ (NR2B3). The transcriptional activity of RXR mainly results from its capacity to act as a cognate partner for other NRs. RXR can engage in 3 types of partnerships, permissive, conditional and non-permissive. Non-permissive heterodimers, such as RXR/VDR (vitamin D receptor) and RXR/TR (thyroid hormone receptor), are activated only by agonists of the partner. Conditional heterodimers, such as RXR/RAR (retinoic acid receptor), are not activated by RXR agonists, but the activity of agonists of the RXR partner receptor is enhanced by RXR agonists (synergistic effect). RXR agonists alone, partner receptor agonists alone or a combination of both can activate permissive heterodimers. Such complexes include heterodimers formed with PPAR (peroxisome proliferator-activated receptor), FXR (farnesoid X receptor), LXR (liver X receptor), and the orphan NR4A group NRs Nur77 and Nurr1. The NR4A subgroup of nuclear receptors includes Nur77 (NR4A1, also known as NGFI-B or TR3), Nurr1 (NR4A2) and Nor-1 (NR4A3). Nur77 and Nurr1 transcriptional activities can be indirectly manipulated through modulation of their heterodimeric partner RXR, using rexinoids such as LG100754, SR11237, BRF110, HX531, HX630 or HX600, or other RXR/NR4A ligands and modulators such as spironolactone, haloperidol, C-DIM12, C-DIM8 and cilostazol.

This invention discloses new compositions and improved utilities of the immodulin peptide class. Sequence extensions to immodulin core sequences disclosed in the invention confer new biological activities useful in treating human disease and cosmetics. Sequence extension of immodulin peptides has been described in PCT/US2020/024828, U.S. Pat. No. 5,519,003/5,783,405/6,165,977/6,262,023/6,342,368/6,423,684/6,855,693/6,933,275/7,393,835/8,536,135/10,369,1919 and references cited therein.

Methods disclosed in this invention include the administration of pharmaceutical compositions containing episynchron peptides to a mammal showing symptoms that may be linked to disease conditions, including but not limited to metabolic and cardiovascular diseases (especially those characterized by some underlying combination of insulin resistance, hyperglycemia, hypertension and hyperlipidemia); immune response to vaccination, cancer progression and metastasis, pulmonary distress and acute kidney injury (AKI) in critical care settings, sepsis, anemia, systemic inflammatory conditions such as shock, post-operative oxidative stress such as after cardiopulmonary bypass or transplant, burns, blunt trauma, pancreatitis, rhabdomyolysis, xenobiotic stresses caused by cocaine, nicotine, alcohol, aminoglycoside antibiotics, cyclosporins, antiviral compounds or chemotherapeutic agents such as platinum compounds or doxorubicin; neuropathic pain and migraine; neurodegenerative diseases such as major depression, Parkinson's, Alzheimer's, Huntington's and ALS/Lou Gehrig's disease; periodontal, orthodontal and orthopedic conditions; immunosuppression phenomena; pathological angiogenesis; impaired wound healing; ototoxicities; autoimmune conditions such as lupus erythematosus, arthritis, psoriasis, colitis, fibromyalgia, and multiple sclerosis; allergic conditions such as rhinitis and its complications, such as nasal hyperreactivity; genetic diseases such as immune insufficiencies; cystinosis, Fanconi's and other conditions affecting mitochondrial respiration; other forms of mitochondrial dysfunction of bioenergetic failure; migraine; pulmonary diseases, especially chronic obstructive pulmonary disease, pulmonary arterial hypertension and asthma; ocular diseases such as cataracts and retinopathies, and conditions caused by infectious agents, including chronic viral infections such as hepatitis, influenza and coronavirus.

In one aspect, the invention provides a modified peptide (i.e. an episynchron) formed by covalent linkage of the moieties “E” and “C-lig” wherein: [a] “E” moiety is a synthetic peptide, 20-60 amino acids in length, comprising an amino acid sequence corresponding to any one of SEQ ID NOs: 1-7, having a lysine residue at or within five amino acid residues of its C-terminus and able to bind at least one isoform of RXR nuclear receptor with submillimolar affinity in vitro; b] “C-lig” moiety is a non-amino-acid carboxylate having a molecular mass of less than about 1000 daltons which is independently capable of binding at least one nuclear receptor isoform with submillimolar affinity in vitro, and [c] said linkage between “E” and “C-lig” is achieved via the formation of a covalent bond between the carboxyl group of “C-lig” and the epsilon amino group of said C-terminal lysine of “E”, said covalent bond formation occurring prior to completion of the amino acid chain elongation steps during peptide synthesis.

In some embodiments the episynchron facilitates the formation of at least one species of RXR heterodimer more efficiently than “E” moiety alone at equivalent submicromolar concentrations. For example, using cultured mammalian cells, a practitioner sufficiently skilled in the art can compare “E” moiety with a modified peptide built from the equivalent “E” moiety covalently linked to a “C-lig” moiety according to the disclosed invention. In some embodiments, said species of RXR heterodimer comprises an RXR isoform and a nuclear receptor isoform selected from a group consisting of: NR1A class, NR1B class, NR1C class, NR1H class, NR2B class and NR4A class.

In other embodiments, at least one additional non-amino-acid carboxylate of mass less than about 1000 daltons is covalently attached to the episynchron. In some embodiments said additional non-amino-acid carboxylate is selected from the group consisting of: oleic acid, eicosapentanoic acid, lauric acid, decanoic acid, lignoceric acid, docosahexanoic acid, 2-hydroxy-2-decenoic acid, phenolic acids, anthraquinones, pentacyclic triterpenoids, retinoic acids, adapalene, bexarotene, tamibarotene, palovarotene, CD1530, CD2665, LG100754, HX600, HX531, HX630, other rexinoids, pirinixic acid, GW7647, GW4064, MBX-8025, GW501516, GW7024, AM580, AC-55649, BMS 453, BMS 493, TTNPB, GIT-27, AZD 9496, LX-093, rhein, proprionic acids, conjugated linoleic acids, keto acids, cinnamic acids, aromatic carboxylic acids, indoleacetic acids, xanthenes, xanthones, fenofibric acid, ciprofibrate, other fibrates, valproic acid, 2-hexyl-4-pentynoic acid, 2,7-dichlorodihydro-fluorescein diacetate, indolyl-carboxylic acids, ibuprofen, artemisinic acid, CY-09, tranilast (MK-341, SB252218) and bromopyruvic acid.

In some embodiments the episynchron is complexed with a metal selected from the group consisting of: ferrous iron, ferric iron, zinc, copper, vanadium, ruthenium, cobalt, titanium, manganese, and calcium.

In some embodiments the episynchron is complexed or co-administered with a natural antioxidant or antimicrobial compound selected from a group consisting of: resveratrol, quercetin, curcumin, ursolic acid, berberine, tannic acid, chitosan compound, menthol, anisic acid, verbenone, linalool, ferulic acid, glycyrrhizic acid, glycyrrhetinic acid, and levulinic acid.

In some embodiments an episynchron described herein is complexed with a glycosaminoglycan or other extracellular matrix component selected from the group consisting of: collagen, iron-binding proteins, gallocyanine, gallates, heparin, heparan sulfate, chondroitin sulfate, keratan sulfate, dermatan sulfate, and hyaluronate.

In a related aspect the invention provides a pharmaceutical composition that includes any episynchron or peptide complex described herein, and a pharmaceutically acceptable excipient.

In another related aspect the invention provides a pharmaceutical composition that includes any episynchron or peptide complex described herein formulated as a microparticle or nanoparticle using methods well known to practitioners in the art. In some aspects, said microparticle or nanoparticle is prepared with compounds and polymers such as polyvynyl alcohol (PVA), various surfactants, metals, carboxymethylcellulose (CMC) or polylactic-co-glycolic acid (PLGA); glycosaminoglycans such as hyaluronate; chitosan compounds such as glycol chitosan, chitosan lactate or any chitosan oligosaccharide; a natural antioxidant or antimicrobial compound such as from the list recited above; or human serum albumin with or without conjugation to a homing peptide or other addressin. As is well known by practitioners in the art, a vast number of protocols have been established for the preparation of microparticles or nanoparticles using the ingredients recited herein.

In yet another related aspect the invention provides a method for treating a subject suffering from immunological, neurological, oncologic, skeletal, reproductive, metabolic or cosmetic dysfunction or perturbation, where the method includes administering to the subject (e.g., a human subject) a therapeutically effective dose of episynchron peptide, peptide complex, or a pharmaceutical composition as described herein. In some embodiments the therapeutically effective dose of the episynchron is from about 0.01 mg/kg/day to about 50 mg/kg/day.

In yet another related aspect, the invention provides an in vitro method for measuring the potency of any immodulin peptide described herein using cultured mammalian cells.

The compositions of the invention may be administered by means that include but are not limited to oral, sublingual, mucosal, intranasal, transdermal, subcutaneous, intravenous, intraarterial, intraperitonial, intramuscular, intrathecal, pulmonary inhalation, vaginal and rectal.

### DETAILED DESCRIPTION

The terms “subject” and “individual”, as used herein, refer to a vertebrate individual, including avian and mammalian individuals, and more particularly to domesticated animals (e.g., dogs, cats, and the like), agricultural animals (e.g., cows, horses, sheep, and the like), and primates (e.g., humans).

The term “treatment” is used herein as equivalent to the term “alleviating”, which, as used herein, refers to an improvement, lessening, stabilization, or diminution of a symptom of a disease or immune perturbation. “Alleviating” also includes slowing or halting progression of a symptom.

As used herein, “co-administration”, “in conjunction with”, “concurrent”, or “concurrently”, as used interchangeably herein, refers to administration of one treatment modality together with or in addition to another treatment modality. As such, “in conjunction with” refers to administration of one treatment modality before, during or after delivery of the other treatment modality to the subject.

The term “episynchron” shall mean a peptide molecule 20-60 amino acids in length prepared by chemical synthesis and comprising an immodulin peptide sequence, modified in comparison to its natural counterpart by substitution of at least one amino acid residue in the natural sequence, and further modified by one or more of the following modifications: (a) extension with a non-IGFBP sequence; (b) stabilization using D-isomers; (c) conjugation to a small bioactive molecule that is not an amino acid; (d) complexation to a metal, transferrin, glycosaminoglycan or helper molecule; or (e) formulation as nanoparticle or microparticle, optionally coated with chitosan compounds and/or human serum albumin. Human serum albumin used herein may additionally be complexed to a homing peptide, which may be preferably, but not limited to, a peptide corresponding to any of SEQ. ID. Nos: 10-16.

“RXR” means retinoid X receptor, and can refer to either the RXR gene or the protein it specifies. “Rexinoid” means a ligand of an RXR receptor. “RXRs” means any of the RXR isoforms, such as RXR-alpha (RXRα), RXR-beta (RXRβ), RXR-gamma (RXRγ), which may form heterodimers with other nuclear receptors (NRs) in their own class, or in other classes.

RXRs are known to form heterodimers with members of several classes of NRs. NRs are classified into seven classes, NR0 through NR6. It is known that classes NR1-NR4 contain heterodimeric partners of RXRs. Each class contains subclasses e.g. class NR1 contains NR1A, NR1B, NR1C, NR1D, NR1F, NR1H and NR11. Members of at least 5 of these subclasses can form heterodimers with RXRs. Members of NR2, NR3 and NR4 classes also contain members capable of forming heterodimers with RXRs (NR2B class contains the RXRs themselves, and they can homodimerize). A note about nomenclature: The term “NR4A class” includes the orphan nuclear receptors NR4A1, NR4A2 and NR4A3, each one of which can heterodimerize with RXRs. NR4A1 can refer to either the NR4A1 gene or the protein it specifies. The protein, in turn, may have one or more common names in the literature (in the case of NR4A1, the names Nur77, TR3 or NGF1-B). RXR receptors can form functional heterodimers with efficiencies that vary with RXR isoform, actual tissue distribution of each nuclear receptor under a given biological context, epigenetic modulators, and many other variables.

Nuclear receptor classes 1 through 4 contain the following members:

| Class | Protein | Class | Protein | Class | Protein |
|---|---|---|---|---|---|
| NR1A1 | TRα | NR1I1 | VDR | NR3A1 | ERα |
| NR1A2 | TRβ | NR1I2 | PXR | NR3A2 | ERβ |
| NR1B1 | RARα | NR1I3 | CAR | NR3B1 | ERRα |
| NR1B2 | RARβ | NR2A1 | HNF4α | NR3B2 | ERRβ |
| NR1B3 | RARγ | NR2A2 | HNF4γ | NR3B3 | ERRγ |
| NR1C1 | PPARα | NR2B1 | RXRα | NR3C2 | MR |
| NR1C2 | PPARβ/δ | NR2B2 | RXRβ | NR3C3 | PR |
| NR1C3 | PPARγ | NR2B3 | RXRγ | NR3CI | GR |
| NR1D1 | REV-ERBα | NR2C1 | TR2 | NR3I4 | AR |
| NR1D2 | REV-ERBβ | NR2C2 | TR4 | NR4A1 | Nur77 |
| NR1F1 | RORα | NR2E1 | TLX | NR4A2 | NURR1 |
| NR1F2 | RORβ | NR2E3 | PNR | NR4A3 | NOR-1 |
| NR1F3 | RORγ | NR2F1 | COUP-TFα | | |
| NR1H2 | LXRβ | NR2F2 | COUP-TFβ | | |
| NR1H3 | LXRα | NR2F6 | COUP-TFγ | | |
| NR1H4 | FXRα | | | | |

In some embodiments, the invention provides for episynchrons that can facilitate the formation of specific kinds of heterodimers. In some embodiments the invention provides for the formation of RXR heterodimers with members of the NR1A class, NR1B class, NR1C class, NR1H class, NR2B class and NR4A class.

This invention provides a modified peptide (“episynchron”) formed by covalent linkage of the moieties “E” and “C-lig” wherein: [a] “E” moiety is a synthetic peptide, 20-60 amino acids in length, comprising an amino acid sequence corresponding to any one of SEQ ID NOs: 1-9, having a lysine residue at or within five amino acid residues of its C-terminus and able to bind at least one isoform of RXR nuclear receptor with submillimolar affinity in vitro; [b] “C-lig” moiety is a non-amino-acid carboxylate having a molecular mass of less than about 1000 daltons which is independently capable of binding at least one nuclear receptor isoform with submillimolar affinity in vitro, and [c] said covalent linkage between “E” and “C-lig” is achieved via the formation of a covalent bond between the carboxyl group of “C-lig” and the epsilon amino group of said lysine of “E”, said covalent bond formation occurring prior to completion of the amino acid chain elongation steps during peptide synthesis.

The invention provides an episynchron according to the above wherein, in some embodiments, said peptide sequence is extended by a biologically active sequence, such as kinase regulator sequence. The kinases thus regulated may be of the PKC class, the mTOR class, or any other class well known in the art.

In some embodiments, the invention provides a therapeutic immodulin peptide complexed with metal, wherein said metal is selected from a group consisting of ferrous iron, ferric iron, zinc, copper, vanadium, ruthenium, cobalt, titanium, manganese, and calcium. In other embodiments, the invention provides a therapeutic immodulin peptide complexed with a component of the extracellular matrix such as collagen, iron-binding proteins or a glycosaminoglycan such as heparin, heparan sulfate, chondroitin sulfate, keratan sulfate, dermatan sulfate, and hyaluronate.

In another aspect, the invention provides a method for treating a mammal showing symptoms of immune dysfunction or perturbation comprising administering to said mammal via intramuscular, subcutaneous, parenteral, transdermal, intranasal, pulmonary, intravenous or intrathecal route of administration a pharmaceutical formulation comprising a therapeutically effective dose of an episynchron peptide according to the invention, and a pharmaceutically acceptable excipient, thereby alleviating said symptoms of dysfunction. “Treatment of a subject suffering from bodily dysfunction” is used to refer to the above. In some embodiments, the episynchron is administered in a therapeutically effective dose of between about 0.001 mg/kg/day to about 50 mg/kg/day.

This invention envisages an in vitro assay method for measuring the biological potency or biodistribution of an immodulin peptide. As will be understood by those of skill in the art, the mode of detection of a diagnostic signal will depend on the exact detection system utilized in the assay. For example, if a fluorescent detection reagent is utilized, the signal will be measured using a technology capable of quantitating the signal from the sample, such as by the use of a fluorometer. If a chemiluminescent detection system is used, then the signal will typically be detected using a luminometer. Methods for detecting signal from detection systems are well known in the art and need not be further described here.

Sequence “identity” and “homology”, as referred to herein, can be determined using BLAST, particularly BLASTp as implemented by the National Center for Biotechnology Information (NCBI), using default parameters. It will be readily apparent to a practitioner skilled in the art that sequences claimed hereunder include all homologous and trivial variants of an immodulin peptide, such as by conservative substitution, extension and deletion in their amino acid sequences. Trivial substitution variants include swapping of an amino acid with another belonging to the same class, without such substitution resulting in any significant and measurable functional improvement. “Classes” of amino acids include positively charged amino acids (arginine, lysine, histidine), negatively charged amino acids (aspartic acid, glutamic acid), aromatic amino acids (tyrosine, phenylalanine, tryptophan), branched chain amino acids (valine, leucine isoleucine) and other natural groupings such as (serine, threonine) and (asparagine, glutamine).

For testing efficacy of pharmaceutical composition containing an immodulin peptide, an effective amount of therapeutic agent is administered to a subject having a disease. In some embodiments, the agent is administered at about 0.001 to about 50 milligrams per kilogram total body weight per day (mg/kg/day). In some embodiments the agent is administered at about 0.001 to about 40 mg/kg/day, e.g., 0.01, 0.015, 0.02, 0.05, 0.1, 0.2, 0.5, 0.7, 1, 2, 4, 5, 7, 9, 10, 15, 20, 25, 30, 35 or another dose from about 0.001 mg/kg/day to about 40 mg/kg/day.

Therapeutic episynchrons may be administered in a nanoparticle, microparticle or nanoemulsion.

As will be understood by those of skill in the art, the symptoms of disease alleviated by the instant methods, as well as the methods used to measure the symptom(s) will vary, depending on the particular disease and the individual patient. All references cited in this document, including patent applications and publications cited therein, are incorporated by reference in their entirety.

### EXAMPLES

### Example 1. C-Terminal Modification of Peptide with Non-Amino Acid Carboxylic Acids of Low Molecular Mass

N-terminal modification of peptides with biotin, or fatty acids such as myristic and palmitic acids, is widely known in the field. Other types of carboxylic acids have rarely been used in this way, largely due to technical difficulties in recovering the correct product. For immodulin peptides, previous disclosures of N-terminal modification using non-conventional compounds include PCT/US2022/018449, PCT/US2021/046814, PCT/US2021/021433 and PCT/US2020/024828, as well as scientific publications (see, for example, Mascarenhas, 2022 and references cited therein). PCT/US2020/024828 teaches methods and compositions based on N-terminal covalent addition of non-amino acid carboxylate small molecules to immodulin peptides, a conjugation step that occurs after assembly of the amino acid part of the chain in synthesis. Sufficient molar ratios of the non-amino acid carboxylate must be used in that process, in order to satisfy not just a reaction with the correctly assembled chain, but also the much more numerous failure sequences (such as deletions) accumulated during chain elongation. For immodulin peptides, which can often be between 35 and 45 amino acid residues in length (and sometimes longer), such failure sequences can significantly raise the cost of identification and recovery of useful product from the reaction mixture. In practice, therefore, the use of the N-terminal addition method disclosed in PCT/US2020/024828 is largely limited by the cost of the said non-amino acid carboxylate reagent, the covalent addition of non-amino acid carboxylates to a C-terminally located lysine in the immodulin peptide sequence. This reaction occurs, near the start of amino acid chain elongation during synthesis. Regardless of the length of the final peptide chain, the option to couple a non-amino acid carboxylate to the epsilon amino group of a C-terminal lysine monomer via a covalent bond obviates the abovementioned technical problems encountered with the method disclosed in PCT/US2020/024828, vastly increasing the options previously foreclosed due to cost. In short, C-terminal addition allows for the addition of more expensive compounds. As described, the invention provides for the addition of non-amino acid carboxylates wherein said molecules can bind a nuclear receptor with sub-millimolar affinity in vitro. There are, however, novel technical issues that limit C-terminal in-synthesis conjugation of such non-amino acid carboxylates. For example, said compounds must be stable enough to survive multiple cycles of synthesis under the harsh conditions typically employed for automated peptide synthesis. For some compounds, if the conjugation is not efficient, the C-terminal monomer may need to be modified and pre-purified prior to use in chain elongation. The general methods disclosed herein are applicable to a number of compounds that are capable of binding a nuclear receptor with sub-millimolar affinity in vitro. In practice, however, it is not always possible to predict in advance which ligands will work satisfactorily, and which will not. None of the nuclear-receptor-binding compounds successfully exemplified here have ever been previously coupled to the C-terminal lysine residue of an immodulin peptide (or, indeed, to the best of the inventor's knowledge, to any other peptide). This invention discloses, for the first time, small non-amino acid carboxylates that can bind a nuclear receptor with sub-millimolar affinity in vitro conjugated to the C-terminus of an immodulin peptide during synthesis.

The peptides were synthesized on ChemMatrix Rink Amide resin, as follows, using a modified Fmoc synthesis protocol with DIC/CI-HOBt coupling, on an APEX 396 automatic synthesizer. For example, using GW7647 as the compound, Fmoc-d-Lys (GW7647)-OH was customized in this way: GW7647 was activated as an NHS-ester and then reacted with Fmoc-d-Lys-OH, which had a free epsilon amine on its side chain. The FMOC-d-Lys (GW7647) wang resin was swollen in dimethylformamide (DMF) for 30 min, treated with 20 v % Piperidine-DMF for 8 minutes to remove the Fmoc protecting group at 50° C., and washed with DMF three times. For the coupling reaction, the resin was added with Fmoc-protected amino acid, CI-HOBt, DIC and NMP. The mixture was vortexed for 20 minutes at 50° C. Afterwards, the resin was washed with DMF once. The cycle of deprotection and coupling steps was repeated until the last biotin residue was assembled. The resin was then washed with DMF, DCM and dried with air. The peptide was cleaved from the resin using a TFA cocktail (95 v % TFA, 2.5 v % water and 2.5 v % TIS) for three hours. Crude peptides were precipitated by adding ice-chilled anhydrous ethyl ether, washed with anhydrous ethyl ether for three times, and dried in vacuo. The purification was performed accordingly by a prep-HPLC. The results of the conjugation experiments show that there is wide variation in conjugation efficiency from compound to compound. As the practicality and cost of synthesis can be dramatically affected when product yield is low, it is therefore not obvious a prior that any untested carboxylic acid should be assumed to be a good candidate for this type of peptide modification. The use of compounds tested here and listed in the table below has never been reported for this kind of peptide modification. It appears that that chance of practical success is low until shown. Once shown, however, the C-terminal modification of an immodulin peptide with a given compound is nearly always highly reproduceable. Some interesting examples are shown in the table below:

| Ligand Class | Compound | CAS No. | MW |
|---|---|---|---|
| PPARα | GW7647 | 265129-71-3 | 502.8 |
| PPARα | Cipro | 52214-84-3 | 289.2 |
| PPARd | GW501516 | 317318-70-0 | 453.5 |
| PPARγ | Capric acid (decanoate) | 1002-62-6 | 194.2 |
| FXR | GW4064 | 278779-30-9 | 542.8 |
| LXR | GW3965 | 405911-17-3 | 618.5 |
| RARβ/γ | adapalene | 106685-40-9 | 412.5 |
| RARα/β | Tamibarotene (AM80) | 94497-51-5 | 351.4 |
| RARα | AM580 | 102121-60-8 | 351.4 |
| RARβ | BMS453 | 166977-43-1 | 380.5 |
| RARγ | palovarotene | 410528-02-8 | 414.5 |
| RXR | Bexarotene | 153559-49-0 | 348.5 |
| RXR | HX531 | 188844-34-0 | 483.6 |
| RXR | HX600 | 172705-89-4 | 438.6 |
| RXR | LG100754 | 180713-37-5 | 396.6 |

Table X1.1. C-terminal modification of immodulin peptides (“E”) with listed “C-lig” moieties.

The ligands of nuclear receptors listed in the table X1.1 above (referred to as “C-lig” moieties) each bind their cognate nuclear receptor with submillimolar affinity in vitro. An episynchron is an immodulin peptide “E” modified at a lysine near its C-terminus by conjugation of a C-lig moiety via a covalent bond at the epsilon amine of said lysine. C-lig moieties are shown in the above list and also include many additional NR ligands well known to those versed in the art. Some examples of episynchrons made by the above method and used in bioassays described in this example are shown in the table X1.2 below:

TABLE X1.2
Examples of episynchrons formed by conjugation of “E” peptide to “C-lig”

| Peptide ID | “E” peptide moiety | “C-lig” moiety | Ligand for |
|---|---|---|---|
| imm3ppa | SDKKGFYKKKQCRPSKGRKRGFCWSVDK | GW7647 | PPARα |
| imm3cip | SDKKGFYKKKQCRPSKGRKRGFCWSVDK | Ciprofibrate | PPARα |
| imm3dec | SDKKGFYKKKQCRPSKGRKRGFCWSVDK | Capric (decanoic) | PPARγ |
| imm3fxr | SDKKGFYKKKQCRPSKGRKRGFCWSVDK | GW4064 | FXR |
| imm3adp | KKGFYKKKQCRPSKGRKRGFCWSVDK | adapalene | RARβ/γ |
| imm3tam | SDKKGFYKKKQCRPSKGRKRGFCWAVDK | tamibarotene | RARα/β |
| imm3ra1 | SDKKGFYKKKQCRPSKGRKRGFCWSVDK | AM580 | RARα |
| imm3bex | SDKKGFYKKKQCRPSKGRKRGFCWSVDK | bexarotene | RXR |
| imm3hx5 | SDKKGFYKKKQCRPSKGRKRGFCWSVDK | HX531 | RXR |
| imm3LG7 | SDKKGFYKKKQCRPSKGRKRGFCWSVDK | LG100754 | RXR |
| imm3K1tam | SLNPEWNETKKGFYKKKQCRPSKGRKRGFCWAVDK | tamibarotene | RARα/β |
| imm3K1K4 | SLNPEWNETFHDAPIGYDKKGFYKKKQCRPSKGRKRGFCWSVD | | control |
| imm3K1 | SLNPEWNETKKGFYKKKQCRPSKGRKRGFCWAVD | | control |
| imm3K4 | HDAPIGYDKKGFYKKKQCRPSKGRKRGFCWAVD | | control |
| imm3AVD | KKQCRPSKGRKRGFCWAVD | | control |
| Imm3SVD | KKQCRPSKGRKRGFCWSVD | | control |

Practitioners versed in the art will recognize the possibility of adding NR ligands to the N-terminus of an episynchron, using the methods for N-terminal modification referenced above and in the examples below. For example, ligand GW4064 can be added to the N-terminus of episynchron imm3bex, to create imm3fxr-bex: [fxr-SDKKGFYKKKQCRPSKGRKRGFCWSVDK-[bex]. This type of construction allows two different ligands to modify transcription within the same cell nucleus simultaneously. Adding the two ligands separately to achieve an identical result in a live mammal would not be possible because of dose-limited toxicities. No peptide carrying two different classes of NR ligand to the nucleus simultaneously has ever been described. Episynchrons thus pave the way for novel interventions in medicine.

Biological effects of episynchrons can be shown in many different biological systems, both in vitro and in vivo. Mascarenhas (2022) provides detailed descriptions of assays for CD169+ macrophage generation and C2C12 myoblast differentiation mediated by immodulin peptides. Examples of the performance of episynchrons in these assays are shown in the tables below. Table X1.3 shows examples of CD169+ macrophage differentiation (and subsequent effects of Th subsets (by co-culture with CD4+naïve T-helper cells). Briefly, macrophage differentiation and polarization assays were done using the THP1-Dual monocyte reporter cell line (Invivogen Inc, San Diego, CA) seeded at 2×10^{e}5 cells per well in 96-well plates and cultured at 37 degrees C. in RPMI-1640 growth medium plus 10% fetal bovine serum and 1% penicillin/streptomycin, then treated for 24 hours with 100 ng/ml Phorbol 12-myristate 13-acetate (macrophage PMA protocol). Peptide (330 or 660 nM) was then added, and incubation continued for an additional 24 hours. Culture supernatants were then assayed for C—C motif chemokine 22 (CCL22), interleukin-10 (IL-10) or transforming growth factor beta (TGFbeta) using Duoset ELISA kits (R&D Systems, Minneapolis, MN). Adherent cells were washed twice with PBS and cells were assayed for immunoreactivity of surface markers CD169, Clec9a and Clec12a using biotin-labeled anti-human antibodies for these markers purchased from Miltenyi Biotec (Auburn, CA) and a streptavidin-horseradish peroxidase/TMB secondary detection reagent. Results were expressed as arbitrary ELISA immunoreactivity units relative to the control peptide (=100). For T-cell differentiation co-culture assays, the THP1-Dual monocyte reporter cell line was treated with PMA for 24 hours as described above, adherent cells were washed twice with PBS and approximately 2×10e5 naïve CD4+ T-cells (Zen-Bio Inc, Durham, NC) were layered on the macrophages in 100 uL RPMI-1640 containing peptide and 15 uL T-activator CD3/CD28 Dynabeads (Thermofisher Scientific, Waltham, MA) and incubated for a further 96 hours. Protein was assayed using the BCA protein assay kit from Thermofisher Scientific (Waltham, MA). AU=arbitrary units (immunoreactivity) per milligram protein: Control 100=peptide imm3K1tam.

| Peptide (0.66 uM)> | imm3K1tam | imm3K1 | imm3K1K4 | imm3K4 | imm3AVD | Buffer |
|---|---|---|---|---|---|---|
| CD169 (AU) | 100 ± 4.7 | 24.3 ± 1.6** | 74.0 ± 3.2**# | 71.1 ± 4.3**# | 22.1 ± 3.2** | 3.1 ± 2.7** |
| CCL22 (ng/ml) | 6.2 ± 0.3 | 1.9 ± 0.1** | 2.8 ± 0.2** | 2.3 ± 0.1**# | 0.6 ± 0.1** | 0.2 ± 0.1** |
| CXCL8 (ng/ml) | 8.9 ± 0.3 | 2.7 ± 0.2** | 4.8 ± 0.8** | 3.7 ± 0.1**# | n.d. | 0.1 ± 0.0** |
| IL-10 (ng/ml) | 17.2 ± 0.7 | 4.7 ± 0.3** | 5.3 ± 0.7** | 4.8 ± 0.6** | 1.3 ± 0.1** | 0.1 ± 0.1** |
| TGF-beta (ng/ml) | Not done | 0.8 ± 0.1# | Not done | Not done | 0.1 ± 0.0 | 0.0 ± 0.0 |
| Treg (FoxP3 AU) | 100 ± 3.5 | 85.4 ± 2.6** | 68.6 ± 8.1** | 66.7 ± 7.6** | 46.5 ± 8.5** | 31.6 ± 5.6** |
| Th17 (IL-17 pg/ml) | 35 ± 7 | 235 ± 33** | 278 ± 30** | 390 ± 27** | Not done | 125 ± 27** |
| Th2 (IL-13 AU) | 100 ± 12 | 120 ± 48 | 188 ± 20** | 140 ± 16* | Not done | 120 ± 16 |

Table X1.3 shows examples of CD169+ macrophage differentiation (and subsequent effects of Th subsets (by co-culture with CD4+ naïve T-helper cells).
**p < 0.01
*p < 0.05 vs imm3K1tam control;
#p < 0.01 vs imm3AVD control.

Table X1.4 shows data from C2C12 myoblast differentiation. C2C12 myoblast cell line (ATCC, Manassas, VA) was cultured in DMEM medium supplemented with 10% FBS and 1% penicillin/streptomycin. For differentiation, cells were cultured in the same medium, except that 10% FBS was replaced by 2% horse serum. Peptides and NR ligands were added at the indicated concentrations and cells were harvested 96 hours later for assay. Cell extracts were assayed for creatine kinase (CK) per mg protein. CK was measured using the ECPK-100 kit from BioAssay Systems (Hayward, CA). Protein was assayed using the BCA protein assay kit from Thermofisher Scientific (Waltham, MA). CK activities and p values were expressed relative to imm3SVD peptide (=100). **p<0.01; *p<0.05.

TABLE X1.4
Data from C2C12 myoblast differentiation assay.

| Peptide (0.66 uM) | Added ligand (2 uM) | Agonist of | CK (units/mg) |
|---|---|---|---|
| None | None | None | 5.2 ± 2.1** |
| imm3SVD | None | None | 100.0 ± 2.9 |
| imm3SVD | GW7647 | PPARα | 163.3 ± 2.0** |
| imm3SVD | GW9578 | PPARα | 185.6 ± 10.5** |
| imm3SVD | Ciprofibrate | PPARα | 109.4 ± 3.2* |
| imm3SVD | GW501516 | PPARδ | 174.2 ± 5.0** |
| imm3SVD | GW3965 | LXR | 67.4 ± 5.2** |
| imm3SVD | Capric (decanoic) acid | PPARγ | 116.1 ± 23.2 |
| imm3SVD | GW4064 | FXR | 165.5 ± 11.9** |
| imm3SVD | adapalene | RARβ/γ | 47.8 ± 29.6** |
| imm3SVD | tamibarotene | RARα/β | 88.0 ± 32.7 |
| imm3SVD | sobetirome | THRβ | 220.7 ± 11.9** |
| imm3SVD | bexarotene | RXR | 124.8 ± 13.2* |
| imm3SVD | HX531 | RXR | 41.1 ± 9.9** |
| imm3SVD | HX600 | RXR | 30.3 ± 16.0* |
| imm3SVD | LG100754 | RXR | 193.9 ± 30.8** |
| imm3bex | bexarotene (C-lig) | RXR | 121.0 ± 6.6* |
| imm3fxr | GW4064 (C-lig) | FXR | 136.8 ± 19.6* |
| imm3ppa | GW7646 (C-lig) | PPARα | 155.9 ± 18.8** |
| imm3bex + imm3fxr | | RXR + FXR | 221.3 ± 34.4** |
| imm3bex + imm3ppa | | RXR + PPARα | 236.8 ± 29.2** |

By addition of an additional NR ligand to the C-terminus of an episynchron using the methods described in Examples 1 and 2 above, it is possible to combine the effects of multiple transcriptional pathways at will. For example, the effects of imm3fxr and imm3bex were combined into a single peptide imm3fxr-bex, wherein GW4064 and bexarotene were added to the N- and C-terminii, respectively, of a peptide of identical sequence, In cell culture assays, imm3fxr-bex performs as predicted from the performance of the combination of imm3fxr and imm3bex episynchrons, an example of which is shown in the table above. As noted previously, however, the in vivo performance of imm3fxr-bex (and other such single-molecule combinations, hereinafter termed “multisynchrons”) is expected to be technically and industrially superior, owing to dose-related and regulatory limitations associated with such combinations.

### Example 2. N-Terminal Modification of Peptide with Non-Amino Acid Carboxylic Acids of Low Molecular Mass

N-terminal modification of peptides with biotin, or fatty acids such as myristic and palmitic acids, has been widely used. This example shows the difficulty in predicting success for this type of modification for other carboxylic acids. The data in this example disclose, amongst other facts: (i) coupling to carboxylic acids (that are not proteinogenic amino acids or biotin) using normal peptide synthesis conditions; (ii) surprising results showing that yields of correctly coupled product (as ascertained by liquid chromatography and mass spectroscopy analysis) varied greatly, even within the same class of compound; and (iii) consistent results for small molecules attached by this method to immodulin peptide or a generic D-tetrapeptide dLys-dAsp-dLys-dPro, with similar efficiencies of coupling to either peptide, thereby demonstrating the generality of the method. Peptides were synthesized according to a common Fmoc/tBu solid phase synthesis strategy well-known in the art. Synthesis may be manual of automated. After the peptide synthesis the resin was divided into batches of 20 umol. Each batch was treated with one of the organic compounds specified in Table X2.1 below. The coupling was carried out using 2 equivalents of the compound, 2.4 equivalents of activator HATU or HCTU, and 4 equivalents of NMM base. The reaction mixture was renewed after 2 hrs reaction time and allowed to react another 4 hrs or overnight. After washing the resin several times with DMF, and subsequently with DCM, the batches were dried. For the cleavage of the peptides from the resin the resins were treated with 1% DTT, 2% water and 3% TIPS in TFA for 3.5 hrs. The cleavage solution was separated from the resin and treated with diethylether/n-pentane (1:1). The resulting precipitate was centrifuged and the pellet washed three times in the same DEE/pentane mixture. The recovered peptide was air dried and stored at −20 degrees C. or further purified by HPLC using a 0-50% acetonitrile gradient, 0.1% trifuoroacetic acid (20 min). The results of the above conjugation experiments show that, both inter-class and intra-class, there is wide variation in conjugation efficiency from compound to compound. As the practicality and cost of synthesis can be dramatically affected when product yield is low, it is therefore not obvious that any untested carboxylic acid should be assumed to be a good candidate for this type of peptide modification. The use of most of the compounds tested here has never been reported for this kind of peptide modification. It appears that that chance of practical success (>80% correct yield, for instance) for each instantiation of this technology is less than 50% until tested.

Table X2.1. N-terminal coupling of non-amino acid carboxylates to immodulin peptides.

TABLE X2-1
N-terminal coupling of non-amino acid carboxylates to immodulin peptides.

| Class | Compound | CAS No. | MW | Yld T4* | Yld IM3* |
|---|---|---|---|---|---|
| fatty acid | oleic acid | 112-80-1 | 282.5 | 44.21% | |
| fatty acid | eicosapentaenoic acid | 10417-94-4 | 302.5 | 66.79% | |
| fatty acid | lignoceric acid | 557-59-5 | 368.6 | 89.20% | |
| fatty acid | decanoic acid | 1002-62-6 | 172.2 | 88.67% | 98.0% |
| fatty acid | docosahexanoic acid | 6217-54-5 | 368.6 | 57.77% | |
| fatty acid | lauric acid | 143-07-7 | 200.3 | 85.14% | 96.7% |
| fatty acid | 10-hydroxy-2-decenoic acid | 14113-05-4 | 186.3 | 44.38% | |
| phenolic acid | ferulic acid | 1135-24-6 | 194.2 | 26.58% | |
| phenolic acid | isoferulic acid | 537-73-5 | 194.2 | 55.80% | 70.2% |
| phenolic acid | Aspirin | 50-78-2 | 180.2 | 56.5% | |
| phenolic acid | valeroyl salicylate | 64206-54-8 | 222.2 | 11.76% | |
| pentacyclic | betulinic acid | 472-15-1 | 456.7 | <1% | |
| anthraquinone | Rhein | 478-43-3 | 284.2 | 50.95% | |
| anthraquinone | Diacerein | 13939-02-1 | 368.3 | 43.2% | |
| Xanthone | 2,7-dichlorodihydro- | 4091-99-0 | 487.3 | 91.2% | |
| proprionic acid | (s)-ketoprofen | 22161-81-5 | 254.3 | 77.86% | |
| proprionic acid | Ibuprofen | 15687-27-1 | 206.3 | 93.42% | 98.0% |
| carboxylic acid | trans-cinnamic acid | 140-10-3 | 148.2 | 93.12% | 81.5% |
| carboxylic acid | (s)-(-)-perillic acid | 23635-14-5 | 166.2 | 27.96% | |
| carboxylic acid | fenofibric acid | 42017-89-0 | 318.8 | 85.67% | 99.9% |
| indoleacetic acid | Indomethacin | 53-86-1 | 357.8 | 87.5% | 85.2%# |
| pentanoic acid | valproic acid | 1069-66-5 | 144.2 | 91.43% | 84.9% |
| alkynoic acid | 2-hexyl-pentynoic acid | 96017-59-3 | 182.3 | | 85.1% |
| indolylcarboxylic | RG-108 | 48208-26-0 | 334.3 | | 74.3%@ |
| Retinoid | all-trans retinoic acid | 302-79-4 | 300.4 | 13.1% | |
| Rexinoid | Bexarotene | 153559-49-0 | 348.5 | 97.09% | 94.4% |

Covalent terminal conjugation of carboxylic acids to peptides.
*Percent yield of correct species by MS for T4 (tetrapeptide) and IM3 immodulin peptide (industrially desirable >80% shown in bold type); #lost p-chlorophenone group (incorrect product); @indole core oxidized by Arg protecting groups (incorrect product).

### Example 3. Administration of Episynchrons to Precise Targets In Vivo

Episynchrons offer, for the first time ever, the tools to choreograph combinations of NR-driven transcriptional programs in target tissues at will. Mechanistic specificity can be optimized further if such mechanistic precision is combined with high precision delivery of dosage forms. One imperative of ultimate specificity in precision medicine i.e. therapeutic concentrations of the drug in the target tissue, combined with much lower concentrations elsewhere in the body of the organism (especially in the plasma) can theoretically yield very wide therapeutic indexes (ratios of efficacy to safety). This is particularly relevant to all patient-administered medications. With the rise of vertically integrated healthcare delivery systems, there is rising interest in remotely administered modalities. A self-administered medicine that possesses a broad therapeutic index to ensure safety is of particular interest in this context. Thus, in order to create ease of episynchron self-administration combined with a broad therapeutic index one might wish to combine (a) mechanistic specificity to a particular body compartment by selecting the relevant subset of NR-driven transcriptional programs—a feature that episynchrons now make possible; (b) convenient self-administration via transdermal or intranasal dosage forms; (c) protection of the peptide in a biodegradable polymer such as PLGA; (d) targeting of the drug at therapeutic levels to a desired location in the body while simultaneously maintaing extremely low levels of the drug elsewhere and in the plasma. Therapeutic peptides can be encapsulated in PLGA microparticles or nanoparticles by a number of protocols widely known in the art. For encapsulation in targeted microparticles containing immodulin peptides, the following protocols are used: Solutions are filter-sterile. HAR buffer contains 4 mg/ml high molecular weight hyaluronic acid, 50 mg/ml D-raffinose and 34 mg/ml D-trehalose. PLGA-M solution contains 70 mg/ml Poly(lactide-co-glycolide, acid terminated) i.e. PLGA-COOH (Cat. #26268-1, Polysciences Inc, Warrington, PA) dissolved in 95 parts acetone, and 5 parts 20% menthol in propylene glycol. Microparticles are assembled as follows: 100 uL peptide solution is added to 50 uL HAR buffer and 50 uL saline buffer. 500 uL PLGA-M solution is then added and the mixture is vortexed thoroughly. 1 ml of 2% polyvinyl alcohol is added, followed by vortexing. The resulting mixture is then added to 50 ml tubes containg 15 ml sterile distilled water and vortexed again. The tube is shaken at room temperature with the cap off in a fume hood for 2-18 hours to allow for evaporation of the solvent. Particles are collected by centrifugation at 3,000 rpm in a Beckman centrifuge for 10 minutes. Particles may optionally be coated by resuspension in 500 uL of 2 mg/ml glycol chitosan (or other chitosan compound) for 30 mins, followed by centrifugation to recover the coated particles. Optionally, particles may be resuspended in 500 uL of 10 mg/ml human serum albumin (HSA) for 30 mins, followed by centrifugation. HSA may be pre-conjugated to a homing peptide. Homing peptides have been described in the scientific literature for decades and are well known in the art. Conjugation to albumin or any other protein via a covalent bond is easily accomplished using off-the-shelf-reagents and known chemistries such as those designed to employ NHS esters in the formation of suitable amide bonds. Finally, the particles are washed 3 times with sterile distilled water and collected by centrifugation. In order to deliver the microparticles, they may be suspended in 10×NENP diluent for transdermal delivery, 1×NENP in saline for intranasal delivery, or saline for sub-cutaneous bolus delivery. 10×NENP is prepared as follows: Base solvent is made by mixing 2 ml Tween-80, 4 ml propylene glycol, 16 ml diethylene glycol monoethyl ether and 16 ml glycerol. The following are dissolved in base solvent: 5 mM each menthol, p-anisic acid and verbenone; 1 mM each linalool, ferulic acid and glycyrrhizic acid. Finally, 25 ml of this solution in base solvent is added to 25 ml of 6 mM sodium acetate pH 5.2 containing 0.9% sodium chloride, and 10 mM levulinic acid. By using biotinylated immodulin peptides with or without PLGA encapsulation, significantly increased delivery to tissues using microparticles suspended in the above diluents has been demonstrated. Biodistribution in rats is scored as a multiple of expected background distribution per milligram protein (administered dose of labelled peptide divided by calculated body protein). Using this metric, the following results were obtained at 2 hours post-administration (times theoretical background). Legend: SQ=naked imm3SVD peptide 50 uL PBS buffer subcutaneous bolus; IN═HSA-coated, PLGA-encapsulated imm3SVD peptide 50 uL 1×NENP-saline; IN/LYMP=HSA*-coated, PLGA-encapsulated imm3SVD peptide 50 uL 1×NENP-saline [where HSA* is pre-conjugated with LYMP1 homing peptide (SGPTCAYGWCAK)];

CIRCULATORY AND LYMPHATIC TISSUES
(times theoretical background):

| Tissue | SQ | IN | IN/LYMP |
|---|---|---|---|
| Plasma | 20 | 22 | 30 |
| Red Blood Cells | 4 | 7 | 11 |
| Thymus | | 17 | 50 |
| Inguinal Lymph Nodes | | 40 | 89** |
| Axillary Lymph Nodes | 30 | 34 | 73** |
| Cervical Lymph Nodes | | 187**# | 373**# |

**p < 0.01 versus plasma;
#p < 0.01 versus axillary and inguinal lymph nodes; (n = 4)

SPLANCHNIC TISSUES (times theoretical background):

| Tissue | SQ | IN | IN/LYMP |
|---|---|---|---|
| Kidney | 43 | 107** | 235** |
| Liver | 29 | 93** | 209** |
| Pancreas | 6 | 48 | 152** |
| Small Intestine | | 103** | 158** |
| Spleen | 6 | 13 | 39 |

**p < 0.01 versus plasma; (n = 4)

OTHER TISSUES (times theoretical background):

| Tissue | SQ | IN | IN/LYMP |
|---|---|---|---|
| Right Lung (accessory lobe) | 15 | 14 | 29 |
| Left Lung | | 21 | 50 |
| Heart | 8 | 106** | 220** |
| Calf muscle (soleus) | 3 | 42 | 73** |
| Calf muscle (EDL) | 3 | 18 | 78** |
| Skin | | 44 | 132** |
| Brain (olfactory bulb) | | 16 | 31 |

**p < 0.01 versus plasma; (n = 4)

These results indicate an interesting biodistribution pattern for immodulin peptides, with an obvious preference for lymphatics, splanchnic organs, muscle and skin. The preference is amplified by the use of the LYMP1 homing peptide, The pharmacokinetics of the naked peptide (SQ injection) dosage form also show that the plasma peak is reached in the first 10 minutes after injection (data not shown). These data, combined with very low levels of peptide retained in plasma and red blood cells, suggests the possibility of highly precise targeting within the body using episynchrons.

Furthermore, transdermal application of horseradish peroxidase-labeled immodulin peptide microparticles (same as in “IN” above, but with 10×NENP as diluent) applied directly on the calf skin (right calf) showed the following result:

| Tissue (2 hrs post- | RIGHT | |
|---|---|---|
| application) Units/mg | SIDE | CONTRALATERAL |
| Right Skin | 15 ± 21 | 5 ± 5 |
| Calf muscle (soleus) | 212 ± 41** | 47 ± 16 |
| Calf muscle (EDL) | 134 ± 56* | 26 ± 6 |

**p < 0.01,
*p < 0.05 versus contralateral control;

### Example 4. Anti-Cancer Actions of RXR Modulators

A375, A549 and HuT78 cancer cell lines was obtained from American Type Culture Collection (ATCC). They were grown in a T-75 flask in DMEM (or IMDM, for HuT78) medium containing 10% fetal bovine serum and penicillin-streptomycin at 37° C. in a humidified, 5% CO₂ incubator. Cells (100 □l, 2,000 cells/well; 5,000 cells/well for HuT78) were plated in a 96-well plate and incubated overnight at 37° C. in a humidified, 5% CO₂ incubator. Next day, 10 ul/well of compounds were added (quadruplicate wells). After 72 hour incubation with the compound, cell viability was measured in a luminometer by addition of 100 □L/well CellTiterGlo reagent (Promega Inc, Madison, WI) and reading chemiluminescence after 10 mins, as recommended by the manufacturer. Peptide imm3A1 (ETFSDVWKLL-KKGFYKKKQCRPSKGRKRGFCW) was added at 0.66 uM and RXR modulators, each at 0.5 uM. The results of this experiment are shown below, expressed as percent survival of cells (untreated=100).

| RXR mod1 | RXRmod2 | A375 | A549 | HuT78 |
|---|---|---|---|---|
| none | none | 100.0 ± 1.8 | 100.0 ± 3.3 | 100.0 ± 3.5 |
| HX531 | none | 89.9 ± 1.6 ₐ | 89.2 ± 4.1 _{b} | 88.1 ± 0.8 |
| HX531 | berberine | 81.5 ± 3.2 ₐₓ | 76.6 ± 2.6 ₐₓ | 71.8 ± 3.6 ₐₓ |
| HX600 | none | 57.1 ± 0.6 ₐ | 60.0 ± 0.6 ₐ | 48.7 ± 2.8 ₐ |
| HX600 | berberine | 55.2 ± 1.0 _{ay} | 57.1 ± 1.6 _{ay} | 39.1 ± 3.6 ₐₓ |

a = p < 0.01,
b = p < 0.05 versus untreated;
x = p < 0.01,
y = p < 0.05 versus no berberine;

### Example 5. Collagen Stimulating Activity of Immodulin Peptides

Immodulin peptides have potential untility in the field of cosmetics. HFF-1 cell line from American Type Culture Collection (ATCC) was grown in DMEM Medium containing 10% fetal bovine serum and penicillin-streptomycin at 37° C. in a humidified, 5% CO₂ incubator. 2,000 cells/well were plated in a 96-well plate and incubated overnight. 10 ul/well of peptides (2 uM final concentration) were added in quadruplicate wells. After 72 hour incubation with the compound, supernatants were collected and immunoreactivity was measured by ELISA using monoclonal anti-COL1A1 antibody (Abcam, Cambridge, MA). * p<0.05, ** p<0.01 vs no peptide.

| Peptide | Sequence | COL1 ng/ml | N-lig | C-lig |
|---|---|---|---|---|
| None | | 0.27 ± 0.12 | | |
| IM3 | KKGFYKKKQCRPSKGRKRGFCW | 0.28 ± 0.23 | | |
| IM3dec | (decanoic)-KKGFYKKKQCRPSKGRKRGFCW | 0.41 ± 0.11* | PPARγ | |
| IM3K9 | AFNSYELGSKGFYKKKQCRPSKGRKRGFCW | 0.57 ± 0.13** | | |
| imm3decK9adp# | SAFNSYELGSDKKGFYKKKQCRPSKGRKRGFCWS | 5.27 ± 0.36** | PPARγ | RARβ/γ |

#multisynchron

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A modified peptide formed by covalent amide bond linkage of the moieties “E” and “C-lig” wherein:
[a] “E” moiety is a synthetic peptide, 20-60 amino acids in length, comprising an amino acid sequence corresponding to SEQ ID NO:8 and having a lysine residue at its C-terminus;
[b] “C-lig” moiety is a nuclear receptor ligand independently capable of binding at least one nuclear receptor isoform with submillimolar affinity in vitro.

2. The modified peptide according to claim 1 wherein said covalent amide bond linkage between “E” and “C-lig” is formed prior to completion of the amino acid chain elongation steps during peptide synthesis.

3. The modified peptide according to claim 1 wherein “C-lig” moiety is selected from a group consisting of tamibarotene, palovarotene, GW7647, AM580, GW3965, GW4064, HX600, GW501516, sobetirome, AC-55649, GW9578 and LG100754.

4. The modified peptide according to claim 1 wherein “C-lig” is tamibarotene.

5. The modified peptide according to claim 1 wherein “C-lig” is palovarotene.

6. The modified peptide according to claim 1 wherein “C-lig” is GW7647.

7. The modified peptide according to claim 1 wherein “C-lig” is AM580.

8. A formulation comprising the modified peptide according to claim 1 and acceptable pharmaceutical excipients.

9. The formulation of claim 8 comprising human serum albumin covalently attached to a homing peptide via a covalent bond wherein said homing peptide comprises an amino acid sequence corresponding to any one of SEQ ID NOs: 10-16.

10. The formulation of claim 8 comprising a natural antioxidant or antimicrobial compound selected from a group consisting of: resveratrol, quercetin, curcumin, ursolic acid, berberine, tannic acid, chitosan compound, menthol, anisic acid, verbenone, linalool, ferulic acid, glycyrrhizic acid and levulinic acid.

11. A method for administering the modified peptide of claim 1 in the treatment of a subject suffering from bodily dysfunction.
